(19) United States
(12) Patent Application Publication
(10) Pub. No.: US 2009/0042946 A1
(43) Pub. Date: Feb. 12, 2009

(54) METHOD AND KIT FOR CONTROLLING BLEEDING

(76) Inventors: Bob M. Moore, II, Nesbit, MS (US); Duane D. Miller, Germantown, TN (US)

Correspondence Address: O''Melveny & Myers LLP;IP&T Calendar Department LA-1118, 400 South Hope Street, Los Angeles, CA, 90071-2899, US

(21) Appl. No.: 12/049,713

(22) Filed: Mar. 17, 2008

Related U.S. Application Data
(63) Continuation of application No. 10/411,479, filed on Apr. 8, 2003, now abandoned.

(51) Int. Cl. A61K 31/4525; A61K 31/164; A61K 31/12; A61P 7/00; A61K 31/335

(52) U.S. Cl. 514/338; 514/627; 514/450; 514/678

(57) ABSTRACT

Methods and kits for controlling bleeding from a disrupted blood vessel, wherein a vanilloid receptor agonist is administered to the site of the disruption of the blood vessel in a quantity sufficient to control the bleeding.

### FIELD OF THE INVENTION

The present invention pertains to the field of chemical agents used to control bleeding from a disrupted blood vessel.

### BACKGROUND OF THE INVENTION

Vanilloid receptors are highly expressed in sensory neurons and in the brain, as well as in non-neural tissues such as the kidney, lung, and spleen. These receptors are coupled to a non-specific membrane channel that is preferentially permeable to calcium and sodium ions. This channel is blocked by ruthenium red but not by conventional ion channel blockers.

Several subtypes of vanilloid receptors have been identified. These subtypes are referred to as VR-1, VR-2, VR-3, and VR-4. The VR-1 receptor has been shown to be highly conserved between mammalian species. Both the human and the rat VR-1 receptor have 838 amino acids and a molecular weight of about 94 kD.

Several compounds have been identified that interact non-selectively to the internal binding domain of the various VR receptor subtypes. Examples of such compounds include capsaicin, resiniferatoxin, and arvinil. Additionally, tetrahydrocannabinol (THC) and other cannabinoids, such as anandamide, cross-react with vanilloid receptors. Naturally occurring vanilloid receptor agonists are terpenoids containing an α,β-unsaturated 1,4 dialdehyde (3-formyl-3 butenal) functionality. About eighty of these compounds have been identified from plants, fungi, arthropods, sponges, and molluscs. VRAs have been found to have antimicrobial activities or are repellent to animals such as mammals. Thus natural VRAs may function as a defense mechanism against microbes and predators.

Vanilloid receptor agonists, referred to herein as “VRA”, such as capsaicin, activate fine afferent nerve fibers used in pain transmission and neurogenic inflammation. This results in sensations ranging from hotness to burning pain. The sensations due to contact with a VRA are followed by loss of further sensitivity to capsaicin, insensitivity to noxious heat and chemical stimuli, and loss of ability to release sensory neurochemicals involved in neurotransmission and in inflammation. These effects form the basis of the potential use of capsaicin as an analgesic and an anti-inflammatory agent.

Capsaicin and other VRAs have been used as topical pain control agents and for controlling chronic itch. Resiniferatoxin has also been used to suppress detrusor instability, an important cause of urinary incontinence. Other VRAs have been found to have varied pharmacological uses, such as reducing blood cholesterol levels or inhibiting platelet aggregation.

It would be of significant importance to determine additional potential pharmacologic or other utilities for vanilloid receptor agonists.

### DETAILED DESCRIPTION OF THE INVENTION

It has been unexpectedly discovered that bleeding from a disrupted blood vessel can be stopped, or blood flow can be significantly reduced, by administration of a vanilloid receptor agonist (VRA) to the site of the blood vessel disruption. Thus, in one embodiment, the invention is a method to control bleeding from a disrupted blood vessel. According to this embodiment, a VRA is administered to the site of the disruption in an amount effective to control the bleeding. In a second embodiment, the invention is a kit containing a VRA and a pharmaceutically acceptable carrier for administration to the site of disruption of a blood vessel so as to control bleeding from the vessel.

The term “control bleeding” is used herein to mean significantly slowing the rate at which blood exits a blood vessel through a disruption in the wall of the blood vessel. Preferably, such slowing of the rate at which blood exits a disrupted vessel is a complete and persistent cessation of bleeding. Less preferably, there is a reduction in blood flow from the site of disruption, which preferably persists for a time which is sufficient to permit other measures to be taken to stop or otherwise control the bleeding. Such measures may include packing a wound, applying a tourniquet, ligating or repairing a bleeding vessel, or other mechanical or chemical measures.

The term “disrupted blood vessel” refers to a blood vessel which has been wounded so that the structural integrity of its wall has been lost and, therefore, blood flowing within the blood vessel prior to the disruption is able to flow to sites external to the blood vessel. Such disruption may be of any form. The wound may be jagged or smooth and may be in any orientation relative to the long axis of the vessel, such as parallel to the long axis of the vessel, perpendicular to the long axis of the vessel, or any orientation in between.

The method of the invention may be used with a blood vessel wound of any size. In accordance with the invention, depending on dosage of VRA applied to a wound, bleeding may be completely and persistently stopped in a completely severed artery up to about 2.5 mm in diameter. In vessels of greater diameter than 2.5 mm, the method of the invention may completely stop all bleeding but more typically the bleeding will continue but at a much reduced rate. This provides time in which other life saving measures may be applied and greatly increases the likelihood of success of such other measures.

The bleeding vessel may be any vessel within the body, including arteries, arterioles, veins, venules, and capillaries. The vessel may be in any part of the body, such as a limb, the trunk, the neck, the head, or within a body cavity such as the peritoneal cavity.

The cause of the wound to the blood vessel is immaterial and may be, for example, due to a laceration by a sharp object such as a scalpel or a knife, a puncture by a projectile such as a bullet, disruption due to an explosive force, or a ripping of a blood vessel due to excessive tensile force. Accordingly, the method is useful in any situation, and especially an emergency situation, to control bleeding from any blood vessel due to any cause.

In accordance with the method of the invention, a VRA is administered to a disrupted blood vessel in an amount effective to control bleeding from the site of disruption. The VRA that is administered in accordance with the invention may be any chemical compound now known or later discovered to be a vanilloid receptor agonist. Compounds that are known to be VRAs, and which are therefore suitable for the method of the invention, include the following.

Capsaicin: N[(4-Hydroxy-3-methoxyphenyl)methyl]-8-methyl-6-nonenamide

Olvanil: N-Vanillyloleoylamide

PPAHV: phorbol 12-phenylacetate 13-acetate 2-homovanillate

Resiniferatoxin: 4-Hydroxy-3-methoxy[(2S,3aR,6aR,9aR,9bR,10R,11R)-3a,3 b,6,6a,9a,10,11,11α-octahydro-6α-hydroxy-8,10-dimethyl-11α-(1-methylethonyl)-7-oxo-2-(phenylmethyl)-7H-2,9β-epoxyazuleno[5,4-e]-1,3-benzodioxol-5 ul]benzeneacetic acid.

Other known VRAs include compounds that have vanillyl-like moieties, such as arvinil, nuvanil, piperine, and zingerone. Other known VRAs include compounds that lack vanillyl-like moieties, such as polygodial, warbuganal, isovelleral, merulidial, cinnamodial, cinnamosmolide, cinnamolide, scalaradial, ancistrodial, scutigeral, aframodial, and β-acardidial. Additionally, cannabinoids which bind as agonists to vanilloid receptors, such as anandamide and arachadonyl glycerol, are suitable for the invention, and to the extent that such cannabinoids are vanilloid receptor agonists, they are included within the VRAs that are suitable for the present invention.

The application of such VRAs may be by any mode by which the VRA will be made to be present at the site of a disruption in a blood vessel in an amount sufficient to control bleeding.

Generally, the mode of application is selected based upon whether the disrupted bleeding vessel is exposed or non-exposed. The term “exposed” as used herein means that the site of bleeding from a vessel is accessible for topical application of a VRA. Typically, bleeding from an exposed vessel results in blood loss from a vessel to a site external to the body. Surgical wounds, such as within the peritoneal cavity during a laparotomy or laparoscopy are also typically exposed wounds even though bleeding from such wounds may not result in blood flow external to the body.

With exposed wounds, the VRA is preferably topically applied to the bleeding site. Suitable modes of application for topical administration include any method by which the VRA may be directly applied to a bleeding site, such as by contacting or otherwise touching the site of disruption, for example by dabbing or painting the site. Alternatively, the application may be by directing a projectile of liquid, such as a mist or jet or stream of liquid, containing the VRA to the site of disruption, for example by spraying or misting the site.

With such topical application, dosage of VRA applied to a site of bleeding is any amount that is effective to control bleeding. Typically, with topical application, there is no maximum dosage applied because any amount applied in excess of that which is necessary for bleeding control will generally not cause systemic effects.

One exception to the above is with exposed wounds that are situated on or near a surface through which significant quantities of VRA can enter the systemic circulation. This may occur with internal surgical wounds or with deep traumatic injury, such as due to an explosive force or a penetrating ballistic wound. In such circumstances, as described below, care should be utilized to minimize the amount of VRA that enters the systemic circulation.

VRAs, when administered systemically in sufficient quantities, may cause complete and irreversible circulatory collapse resulting in rapid death. Thus, it is imperative, when topically treating an exposed internal wound to apply the VRA in as narrow a field as possible and to preferably apply no more VRA than is necessary for its beneficial bleeding-control effects. Thus, wide-area spraying of the peritoneal cavity is not preferred, although, as discussed below, such broad spraying may be safely utilized in certain situations. Rather, if possible, a jet or stream spray or a touch application of VRA is preferred when utilizing the method of the invention to control bleeding from an exposed internal blood vessel.

A similar situation exists when using the method of the invention to control bleeding from a non-exposed site. Examples of such non-exposed sites include bleeding into a closed peritoneal or pleural space, or bleeding within an organ such as the lungs, liver, or the central nervous system. In these instances, a VRA may be administered systemically such as by intravascular injection, nasal spray, or intraperitoneal infusion. As with exposed bleeding sites where a considerable risk of systemic introduction exits, it is preferred to administer as low a dose as is necessary to control bleeding.

Additionally, with systemic administration of a VRA, it is preferred to use a VRA that is not a “rapid onset VRA”. At high systemic concentrations, VRAs act as neurotoxins that cause a sustained depolarization of nerve cells and an opening of calcium channels that result in a depletion of intracellular calcium and a shutdown of oxidative respiration. Such catastrophic effects most readily occur with the administration of rapid onset VRAs and less readily with “slow onset VRAs”. Rapid onset VRAs are those that open calcium channels immediately whereas slow onset VRAs produce a prolonged or delayed opening of these channels. Thus, slow acting VRAs are preferred for systemic administration. Rapid acting VRAs may be used, however, great care must be taken so that the dose administered is less than that which will injure or kill the subject.

Rapid and slow onset VRAs may be distinguished by any test by which the immediacy of action of a test VRA may be determined. For example, one test to determine whether a VRA is rapid or slow onset is by determining the rate at which a test VRA causes calcium influx into a neuron. This may be performed by labeling a cell with a fluorescent dye sensitive to the intracellular calcium concentration. Upon addition of a test compound to a medium containing calcium ions and fluorescent-labeled neurons, the rate of calcium influx into the cells may be determined. By observing the onset of fluorescence following exposure to a VRA, the VRA may be classified as rapid or slow onset.

Similarly, tests to determine rate of depolarization may be used to determine whether a VRA is rapid or slow onset. One example is such a test is a patch clamp test. A small hole is made in the cell membrane and the difference in membrane potential before and after exposure to a test VRA is determined. By observing the presence of immediate or delayed depolarization, VRAs may be classified as rapid or slow onset.

Examples of rapid onset VRAs include capsaicin and arvinil. Resiniferatoxin is a slow onset VRA. Thus, resiniferatoxin is preferred over capsaicin or arvinil for systemic administration, although capsaicin or arvinil may be systemically administered if care if taken to avoid overdose.

In another embodiment, the invention is a kit which is useful for controlling bleeding from a disrupted blood vessel, in accordance with the method described above. The kit of the invention contains a container housing a chemical compound which is a VRA and a pharmaceutically acceptable carrier. The kit further includes an applicator for administering the VRA to the disrupted blood vessel. The applicator may administer the VRA by touching or otherwise contacting the site of disruption. Thus, for example, the applicator may be a dabber or a roll-on ball. In another alternative, the applicator may administer the VRA by releasing a projectile of liquid containing the VRA to the site of disruption. Thus, for example, the applicator may be a dropper or a mister. Preferably, the kit further contains written instructions for administering the VRA to a disrupted blood vessel in order to control bleeding from the vessel.

The method and kit of the invention have an important utility in the rapid control of bleeding, especially bleeding due to traumatic disruption of a blood vessel. Thus, it is conceived that the invention will be useful in the management of a variety of traumatic injuries, such as due to automobile accidents and lacerations by sharp objects or projectiles. Because of the rapid control of bleeding provided by the invention, the invention is useful in the management of injuries sustained by groups of individuals, such as due to an explosive device. The invention is further useful in controlling bleeding that occurs during surgery, such as in the peritoneal or pleural cavities or in the central nervous system.

The method of the invention may be practiced in humans and other mammals, such as veterinary patients including companion animals like dogs and cats, and farm animals like cows, horses, pigs, goats, sheep, and llamas, and zoo animals. The vanillin receptor is well conserved between species and thus the examples below, illustrating the invention in mice, are indicative of results that one skilled in the art would expect to be obtained in other mammalian and conceivably in other vertebrate species.

The invention is further described in the following illustrative examples.

### EXAMPLE 1

Approximately 4 to 6 week old white mice weighing about 20 grams were anesthetized using 50 microliters of anesthetic (ketamine+xylazine) intraperitoneally. The area near the inguinal region (1.75 cm proximal to the ankle) was shaved and a 0.5 to 1 cm incision was made such that the left femoral artery was severed. The incision was washed with normal saline. Control animals were treated with saline while test animals were treated with a VRA (alvinil) (approx. 500 microliters of a 0.8 mmol solution) administered topically with a cotton-tipped applicator soaked with the VRA solution over the wound. The applicator was then removed after 5 seconds and the wound was evaluated for signs of hemorrhage.

### EXAMPLE 2

In the control mice, the wound was continuously flushed with saline. The animal exhibited signs of shock about 3 minutes post wounding. After 5 minutes, a second incision was made in the femoral artery above the previous cut and essentially no blood was observed in the second incision. Euthanasia with saturated KCl via heart puncture indicated that the heart was essentially depleted of blood.

### EXAMPLE 3

In the treatment group of animals, the wound was treated with arvinil by swab application, then flushed once with saline. No bleeding was observed following the saline flush. No signs of shock were exhibited during the course of the experiment. After 10 minutes, a second incision was made in the femoral artery above the previous cut resulting in severe hemorrhage. Application of arvinil to this second cut resulted in cessation of bleeding. Euthanasia with saturated KCl via heart puncture indicated that the heart was fully perfused.

### EXAMPLE 4

The protocol of Example 1 was repeated except that mice were pretreated intraperitoneally with 500 units/kg of sodium heparin fifteen minutes before severing the femoral artery and application of the cotton applicator containing the VRA was for 30 seconds. As in Example 3, mice treated with the VRA survived the wound and did not exhibit signs of shock. A second incision in a more proximal portion of the femoral artery resulted in profuse bleeding leading to death.

In control animals pretreated with heparin, severing of the femoral artery not followed with treatment with a VRA resulted in rapid shock leading to death by exsanguination.

These results establish that the method of the invention successfully controls bleeding from a disrupted blood vessel, even in a patient that has been treated with anti-coagulants. Thus, the invention is useful to control bleeding even in patients who are receiving anti-coagulant therapy and in surgical patients or other patients who have been administered anti-coagulants, such as to maintain the patency of blood vessels for intravenous infusion.

Further modifications, uses, and applications of the invention described herein will be apparent to those skilled in the art. It is intended that such modifications be encompassed in the claims that follow.

1. A method for controlling bleeding from a disrupted blood vessel comprising administering to the site of the disruption a vanillin receptor agonist (VRA) in an amount effective to control the bleeding.

2. The method of claim 1 wherein the administration is by topical application.

3. The method of claim 2 wherein the application is by directing a projectile of liquid containing the VRA to the site of disruption.

4. The method of claim 2 wherein the application is by contacting the site of the disruption with an applicator containing the VRA.

5. The method of claim 2 wherein the blood vessel is an arterial blood vessel.

6. The method of claim 2 wherein the blood vessel is a venous blood vessel.

7. The method of claim 2 wherein the VRA is selected from the group consisting of capsaicin, olvanil, PPAHV, resiniferatoxin, arvinil, nuvanil, piperine, zingerone, polygodial, warbuganal, isovelleral, merulidial, cinnamodial, cinnamosmolide, cinnamolide, scalaradial, ancistrodial, scutigeral, aframodial, β-acardidial, and a cannabinoid.

8. The method of claim 7 wherein the VRA is selected from the group consisting of capsaicin, olvanil, PPAHV, resiniferatoxin, arvinil, nuvanil, piperine, zingerone, polygodial, warbuganal, isovelleral, merulidial, cinnamodial, cinnamosmolide, cinnamolide, scalaradial, ancistrodial, scutigeral, aframodial, and β-acardidial.

9. The method of claim 8 wherein the VRA is selected from the group consisting of capsaicin, olvanil, PPAHV, resiniferatoxin, arvinil, nuvanil, piperine, and zingerone.

10. The method of claim 9 wherein the VRA is arvinil.

11. The method of claim 2 wherein the disrupted blood vessel has a cross-sectional diameter, before disruption, of 2.5 mm or less.

12. The method of claim 2 wherein the disrupted blood vessel has a cross-sectional diameter, before disruption, of more than 2.5 mm.

13. The method of claim 2 wherein the disruption of the vessel is by surgical incision.

14. The method of claim 2 wherein the bleeding is from a patient that has previously been administered an anti-coagulant.

15. The method of claim 1 wherein the administration of the VRA is systemic.

16. The method of claim 15 wherein the VRA is a slow onset VRA.

17. The method of claim 16 wherein the VRA is resiniferatoxin.

18. A kit for controlling bleeding from a disrupted blood vessel comprising a container housing a vanillin receptor agonist (VRA), a pharmaceutically acceptable carrier, an applicator for topically administering the VRA to the disrupted blood vessel, and written instructions for administration of the VRA to the disrupted blood vessel.

19. The kit of claim 18 wherein the VRA is selected from the group consisting of capsaicin, olvanil, PPAHV, resiniferatoxin, arvinil, nuvanil, piperine, zingerone, polygodial, warbuganal, isovelleral, merulidial, cinnamodial, cinnamosmolide, cinnamolide, scalaradial, ancistrodial, scutigeral, aframodial, β-acardidial, and a cannabinoid.

20. The kit of claim 19 wherein the VRA is selected from the group consisting of capsaicin, olvanil, PPAHV, resiniferatoxin, arvinil, nuvanil, piperine, zingerone, polygodial, warbuganal, isovelleral, merulidial, cinnamodial, cinnamosmolide, cinnamolide, scalaradial, ancistrodial, scutigeral, aframodial, and β-acardidial.

21. The kit of claim 20 wherein the VRA is selected from the group consisting of capsaicin, olvanil, PPAHV, resiniferatoxin, arvinil, nuvanil, piperine, and zingerone.

22. The kit of claim 21 wherein the VRA is arvinil.

23. The kit of claim 18 wherein the applicator administers the VRA upon touching the applicator to the disrupted blood vessel.

24. The kit of claim 18 wherein the applicator administers the VRA by releasing a projectile of liquid containing the VRA to the disrupted blood vessel.
